(19) United States
(12) Patent Application Publication
(10) Pub. No.: US 2017/0172444 A1
(43) Pub. Date: Jun. 22, 2017

(54) Measurement Device for Scalp Electrode Placement

(71) Applicant: Burlin Larry Brewer, Providence Village, TX (US)

(72) Inventor: Burlin Larry Brewer, Providence Village, TX (US)

(73) Assignee: First Sleep, LLC, Providence Village, TX (US)

(21) Appl. No.: 14/971,914

(22) Filed: Dec. 16, 2015

(51) Int. Cl. A61B 5/0476; A61B 5/00

(52) U.S. Cl. CPC A61B 5/0476; A61B 5/6841

(57) ABSTRACT

A flexible measuring device having an elongated member of a fixed arc length has holes at intervals along the body corresponding with the International 10-20 system of electrode placement. The fixed arc length may be of any length corresponding with pediatric and adult head sizes. There may be additional demarcations located along the fixed arc length labeled with standard American or metric units for reference. The flexible measuring device reduces inaccuracy with electrode placement for Electroencephalography (EEG) tests and experiments and can also provide an alternative, low-cost method of measurement for these offices or laboratories.

### CROSS-REFERENCE TO RELATED APPLICATIONS

The present application claims priority to U.S. Provisional Patent Application Ser. No. 62/093,299, filed Dec. 17, 2014, the disclosure of which is hereby incorporated in its entirety at least by reference.

### BACKGROUND OF THE INVENTION

1. Field of the Invention

The present invention relates generally to the technical field of medical measuring devices. More particularly, the present invention relates to a measurement device used in identifying and marking the locations of scalp electrode placement sites used in Electroencephalography (EEG) tests or experiments.

2. Description of Related Art

The International 10-20 System is an internationally recognized method to describe the placement of scalp electrodes at specific intervals along the human head in the context of an EEG test or experiment, ensuring standardized reproducibility over the course of a study of a single subject as well as across subjects.

The International 10-20 System is named for the intervals at which the electrodes should be placed along the scalp; the distances between adjacent electrode sites are either 10% or 20% of the total front-to-back or right-to-left distance of the skull. Each electrode site is assigned a letter to identify the corresponding lobe along with a number or another letter to identify the hemispheric location on the head. The letters F, T, C, P, and O stand for the frontal, temporal, central, parietal, and occipital lobes, respectively. Even numbers (2, 4, 6, 8) refer to the right hemisphere and odd numbers (1, 3, 5, 7) refer to the left hemisphere. The locations of the electrode sites are determined by measurements from four standard anatomical landmarks on the skull: the nasion (just above the bridge of the nose), the inion (the lowest point of the skull at the back of the head, normally indicated by a prominent bump), and the pre-auricular points anterior to each ear. “Z” (zero) refers to the mid-line (or “Z” line) arc that runs longitudinally from the nose over the head and to the base of the skull, connecting the nasion and the inion. Although not within the normal nomenclature of the International 10-20 System, this document will refer to the “Y” Line as the center arc that runs from ear to ear and crosses perpendicularly to the “Z” Line, connecting the pre-auricular points located at each ear.

Until now, the placement of electrodes on the scalp according to the International 10-20 System has been ascertained by mainly one of two methods: (1) A laboratory technician makes measurements using a flexible measuring tape, such as that used by a seamstress or tailor, and calculating the “10-20” points; or (2) Electrode caps are employed to record the electrical activity from the scalp.

While electrode caps provide a quick means of placing electrodes on a subject's scalp, a single cap will not fit everyone; a laboratory may need several caps accommodate various head sizes, but even then there remains head circumferences outside the range of standard caps. Another important factor to consider is the cost of electrode caps; compared to the traditional method, the cost of obtaining many electrode caps, which are not guaranteed to accommodate every head size, may not be viable for certain laboratories.

In contrast, the method of a laboratory technician using a flexible tape measure is cost effective and easily customizable. However, it allows for error in electrode placement, can be time-consuming, and confusing for those who are new in the field.

Therefore, there exists a need for a relatively low cost, simple, and accurate device to facilitate scalp electrode placement to fill the gap between the two, standard methods for EEG tests or experiments.

### SUMMARY OF THE INVENTION

In one embodiment of the present invention a measuring device configured to aid in electrode placement for Electroencephalography tests or experiments is provided comprising an elongated member having a fixed arc length, said elongated member including at least one measuring edge; and at least five holes for electrode placement, extending along the length of said elongated member at fixed intervals.

In one embodiment, said fixed arc length is greater than or equal to 15 centimeters. In another embodiment, said fixed arc length is less than or equal to 60 centimeters. In one embodiment, said five holes at said fixed intervals are located at 10%, 30%, 50%, 70%, and 90% of the said fixed arc length. Furthermore, said measuring device is constructed from a non-rigid material.

In another aspect to the invention, a device for measuring locations on a scalp to place electrodes, the device is provided comprising an elongated member having a fixed arc length, and at least two holes for electrode placement.

In one embodiment, said fixed arc length is less than or equal to 60 centimeters. In another embodiment, said at least two holes are located at 10% and 90% of said fixed arc length. In one embodiment, the device further comprises an additional set of holes at set intervals, said additional set of holes are located between the said at least two holes. In yet another embodiment, said measuring device is constructed of a non-rigid material. In one embodiment, said measuring device is constructed from a thermoplastic material. In another embodiment, wherein said elongated member comprises a set of measuring indicia with corresponding labels referencing American or metric units for said fixed arc length.

In yet another aspect to the invention, a measurement device for electrode scalp placement is provided comprising a flexible body member comprising an elongated portion having a first width, a pair of distal ends having a second width, and at least one measurement portion having a third width; wherein said flexible body member includes a fixed arc length, and said at least one measurement portion includes a set of measuring indicia with corresponding labels referencing American or metric units for said fixed arc length; a pair of holes for electrode placement, said a pair of holes positioned on said pair of distal ends.

In one embodiment, the device further comprises an additional set of holes at set intervals, said additional set of holes are located between said pair of holes on said elongated portion. In another embodiment, said fixed arc length is less than or equal to 60 centimeters. In one embodiment, each hole of said pair of holes is located at 10% and 90% respectively of said fixed arc length. In yet another embodiment, said additional set of holes are positioned at fixed intervals located at 30%, 50%, and 70%, of the said fixed arc length. In one embodiment, said flexible body member is constructed from a thermoplastic material. In one embodiment, said first width is greater than said second width and less than said third width. In another embodiment, said pair of holes and said additional set of holes correspond with an International 10-20 system of electrode placement.

A complete understanding of the invention will be obtained from the following description when taken in connection with the accompanying drawing figures wherein like reference characters identify like parts throughout. However, these embodiments should not be construed as limitations on the scope of any embodiment, but as exemplifications of various embodiments thereof. Many other variations are possible within the teachings of the various embodiments. Thus, the scope should be determined by the appended claims and their equivalents, and not by the examples given.

### BRIEF DESCRIPTION OF DRAWINGS

It is to be understood that the drawings are to be used for the purposes of exemplary illustration only and not as a definition of the limits of the invention. Throughout the disclosure, the word “exemplary” is used exclusively to mean “serving as an example, instance, or illustration.” Any embodiment described as “exemplary” is not necessarily to be construed as preferred or advantageous over other embodiments. Referring to the drawings in which like reference character(s) present corresponding part(s) throughout:

FIG. 1 is a diagram showing electrode placement sites according to the International 10-20 System for electrode placement;

FIG. 2 is an exemplary perspective view of the general structure of a measurement device according to the preferred embodiment of the present invention;

FIG. 3 is an exemplary top plan view of the general structure of the measurement device;

FIG. 4 is an exemplary top perspective view of the measurement device as it is formed for practical usage;

FIG. 5 is an exemplary bottom perspective view of the measurement device as it is formed for practical usage;

FIG. 6 is an exemplary top view of the measurement device as it is formed for practical usage;

FIG. 7 is an exemplary front view of the measurement device as it is formed for practical usage; and

FIG. 8 is an exemplary right side view of the measurement device as it is formed for practical usage.

### DETAILED DESCRIPTION OF THE INVENTION

For a thorough understanding of the present disclosure, reference is to be made to the following detailed description. Although the present disclosure is described in connection with exemplary embodiments, the present disclosure is not intended to be limited to the specific forms set forth herein. It is understood that various omissions and substitutions of equivalents are contemplated as circumstances may suggest or render expedient, but these are intended to cover the application or implementation without departing from the spirit or scope of the present disclosure. Further, it will nevertheless be understood that no limitation in the scope of the disclosure is thereby intended, such alterations and further modifications in the figures and such further applications of the principles of the disclosure, as illustrated therein being contemplated as would normally occur to one skilled in the art to which the disclosure relates. Also, it is to be understood that the phraseology and terminology used herein is for the purpose of description and should not be regarded as limiting. Further, reference herein to “one embodiment” or “an embodiment” means that a particular feature, characteristic, or function described in connection with the embodiment is included in at least one embodiment of the disclosure. Furthermore, the appearances of such phrase at various places herein are not necessarily all referring to the same embodiment. The terms “a” and “an” herein do not denote a limitation of quantity, but rather denote the presence of at least one of the referenced item.

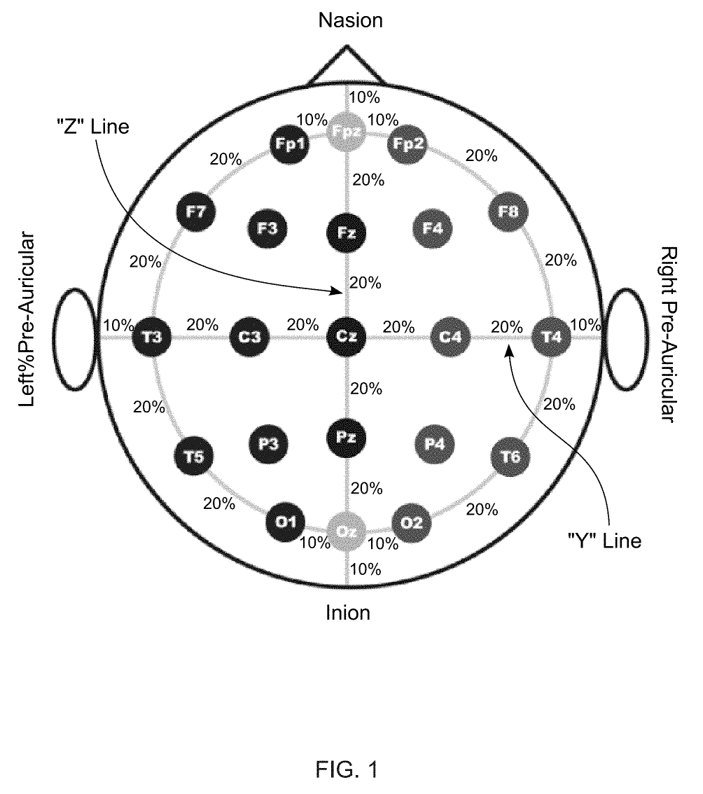

The present disclosure provides a device for measurement adopting the International 10-20 System of electrode placement (FIG. 1) for Electroencephalography (EEG) tests and experiments in a simple device to reduce cost and error in measurement. The present disclosure's device allows measurers to more directly, accurately, and efficiently make a measurement of a scalp. An example of the use of proposed measuring device could be for laboratories or doctor's offices engaged in sleep studies or other neurological study, where EEG tests and experiments are routinely carried out.

Currently, measurers either use a combination of calculators with a standard, flexible measuring tape or costly “caps” that come only in standard sizes. Electrode placement is critical to lab results and to providing useful information to subjects or patients. Therefore, relying on measurements taken by a standard measuring tape might not only lead to inaccuracy in electrode placement, but also inaccuracy in the results obtained. The electrode caps may not fit all scalps accurately and require the laboratory or office to have many in stock for patients or subjects. In conclusion, there are many circumstances at which human error may distort the accuracy of the information measured and there are instances where it is impractical and costly to use electrode caps. Therefore, a new device or method that could eliminate some sources of error could allow for the more direct, efficient and accurate measurement.

The present invention relates to a portable measuring device for electrode placement on a scalp in Electroencephalography (EEG) tests or experiments. The measuring device, comprising a flat, flexible body of a predetermined arc length corresponding with standard scalp measurements and graduated with holes along the length of the body. The graduated holes are at intervals determined by the International 10-20 System of measurement for electrode placement. Accordingly, there is a hole located at 10%, 30%, 50%, 70%, and 90% of the length of the main body. The predetermined arc length varies in size within the range of common scalp arc lengths. The measuring device may additionally include measuring indicia on a measuring edge extending along a finite length of the device with labels corresponding to American or Metric units. The present invention will be described in detail below.

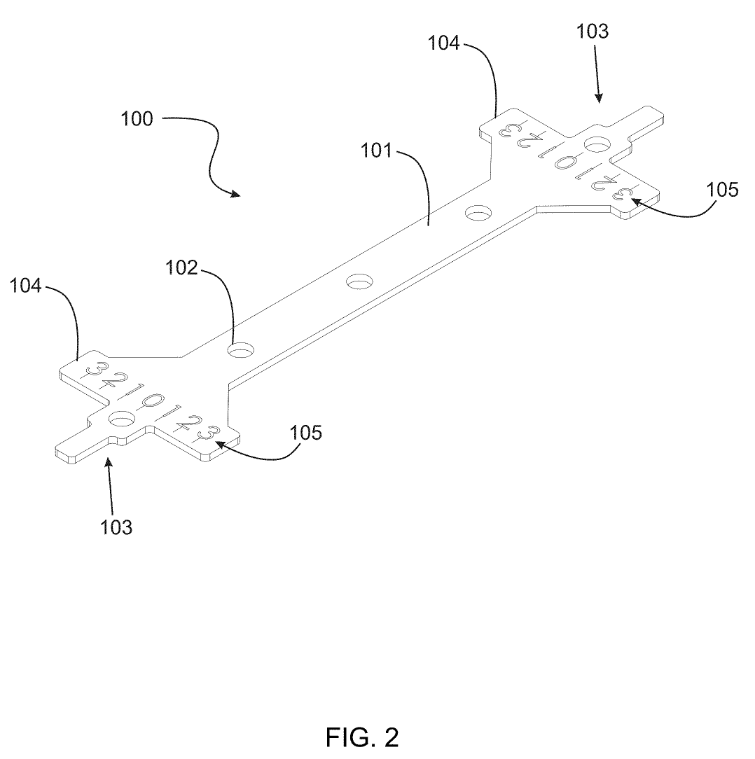

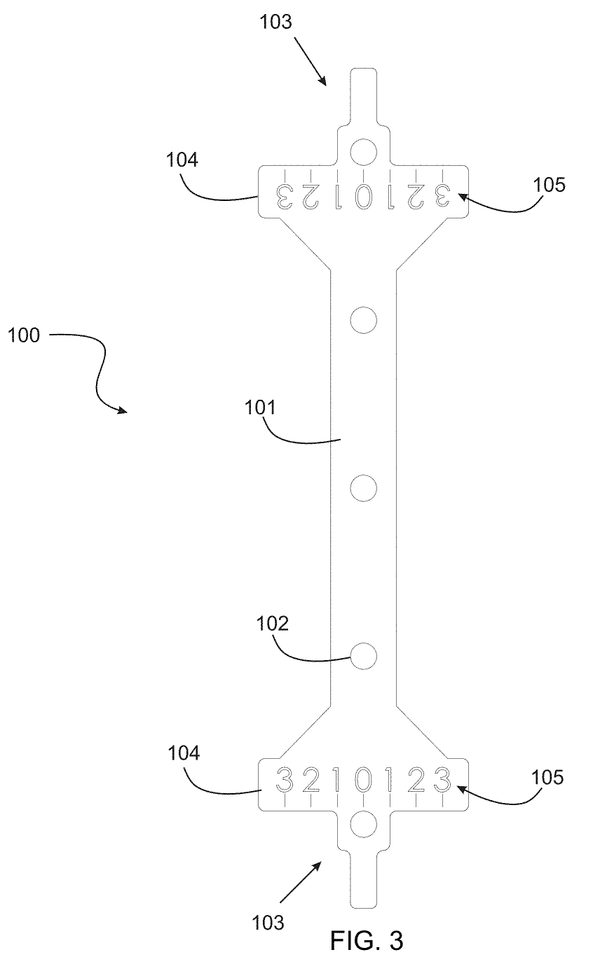

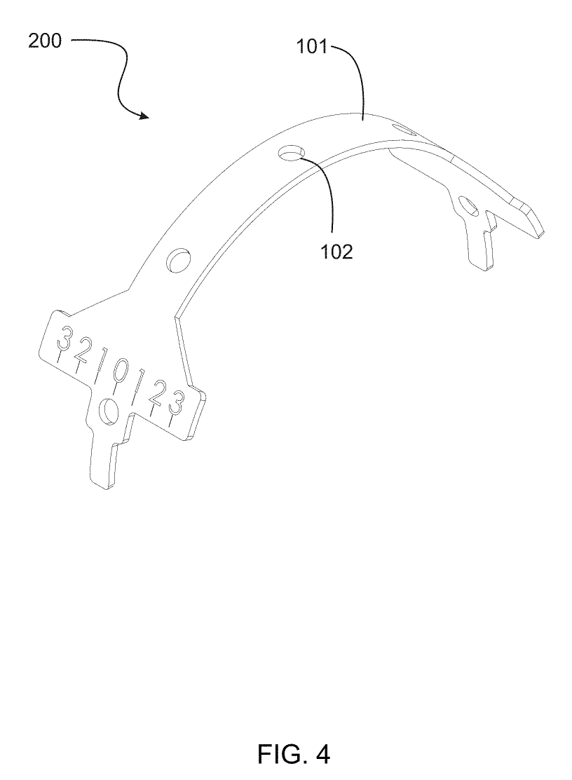

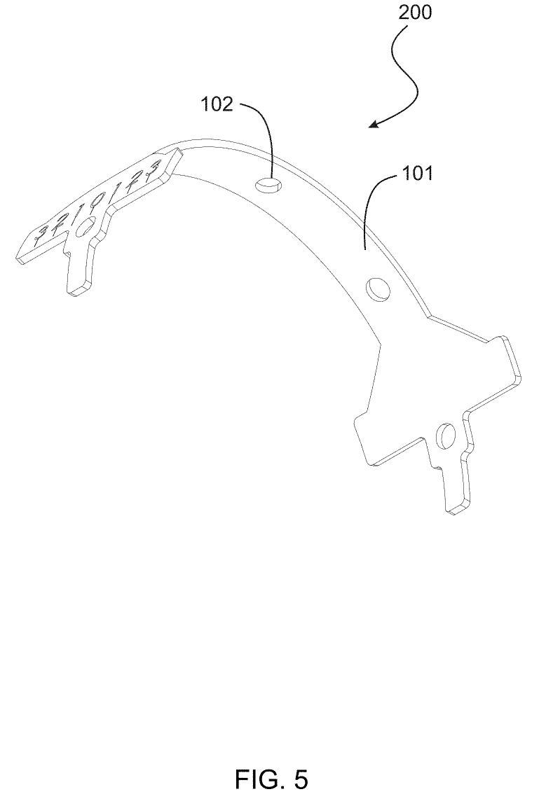

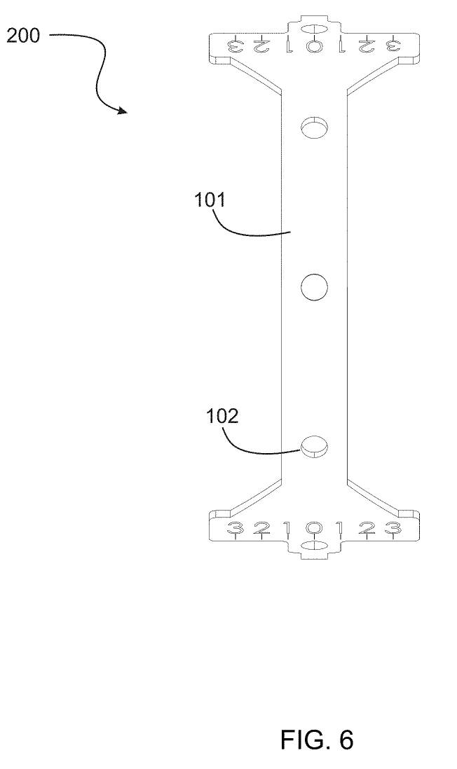

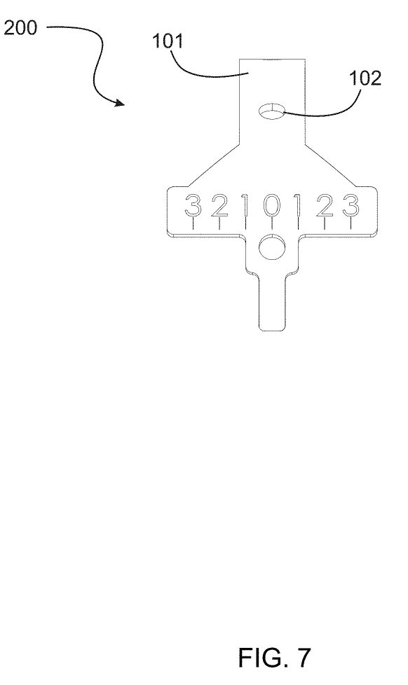

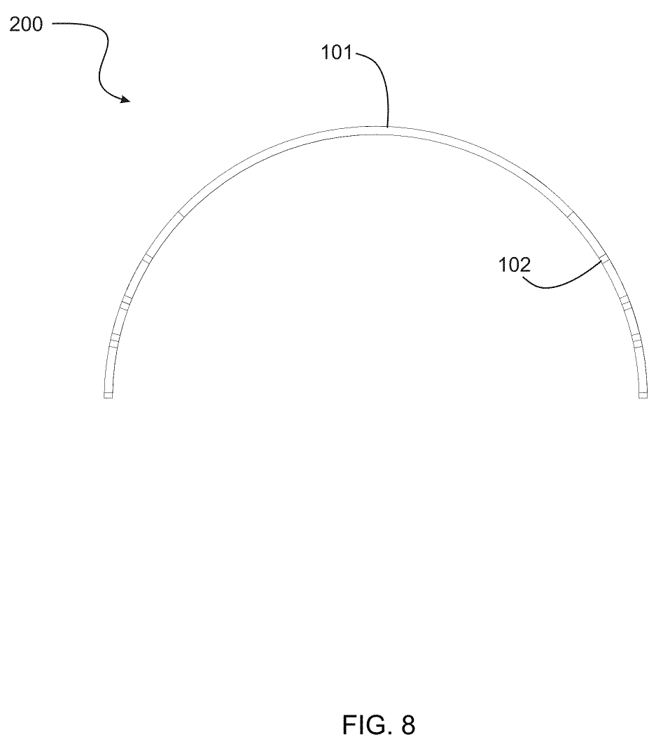

FIGS. 2 through 8 show a single-piece measurement device 100 according to the preferred embodiment of the present invention. FIGS. 2 and 3 illustrate plan views of the general structure of the measurement device, whereas FIGS. 4 through 8 are representative of how the measurement device is physically formed for practical usage.

As most easily seen in FIGS. 2 and 3, the measurement device comprises a main body 101 formed from a thin length of flexible material with holes 102 arranged linearly along the length of the main body, wherein said holes are further centered with respect to the width of the main body and located at appropriate, evenly spaced intervals in accordance with a specific arc measurement that coincide with the International 10-20 System for electrode placement. Accordingly, there is a hole located at 10%, 30%, 50%, 70%, and 90% of the length of the main body.

The main body is substantially the same width over its length except for at two reduced-width portions 103 and two measurement portions 104, all of which are integrally formed with the main body. The two reduced-width portions are positioned adjacent the distal sides of the holes at the 10% and 90% locations, respectively, forming the distal ends of the main body. The two measurement portions are positioned adjacent the proximal sides of the holes at the 10% and 90% locations, respectively, or towards the center of the main body. The measurement portions are preferably formed as wing-like tabs that protrude perpendicularly from the length of the main body in both directions, wherein each tab is equally distributed on each flank of the main body. As can be seen in the drawings, the result of the precedingly described configuration is that the measurement device is symmetrical both lengthwise and widthwise.

Measurement means 105 are provided on one surface of each measurement portion, such as a ruler with numerical markings and tick marks indicating units of distance measured laterally from the center of the main body. Such measurement means may be printed or engraved onto the surface of the measurement device.

Characteristics of the preferred embodiment will now be described in reference to FIGS. 4 through 8. According to the preferred embodiment of the present invention, the measurement device is physically formed such that it generally takes on a horseshoe appearance 200 in its resting position, much like a head band that is commonly worn to hold back long hair. The measurement device is shaped this way in order to form-fit to the contours of the human head, allowing accurate placement of the holes in the main body over the scalp for locating electrode placement sites. It is because of the variability in human head shape and the need for formfitting engagement with the scalp that the measurement device must be suitably flexible. The thin length of flexible material which constitutes the main body of the measurement device is therefore preferably a flexible thermoplastic.

Various versions of the measurement device are categorized according to the dimension of length, in centimeters that corresponds to specific arc measurements of the human head, i.e., the arc lengths of both the “Z” Line and the “Y” Line (FIG. 1). To further account for variability of head circumference, and thus the arc length of the “Z” and “Y” lines, it is contemplated that measurement device need only be manufactured with arc lengths ranging from 20.0 cm to 50.0 cm to accommodate most pediatric and adult subjects.

Other dimensions and ranges of dimensions of other aspects of the measurement device are contemplated. The holes in the main body may be 1.0 cm in diameter. The measurement device can be manufactured with thicknesses ranging from 1/32 in. to ⅛ in. over the entire length of the main body, and widths ranging from 2.0 cm to 2.5 cm over the length main body except for at the two reduced-width portions and two measurement portions. The reduced-width portions may be 1.0 cm wide. In correspondence with the 20.0 cm to 50.0 arc length of the measurement device, it is contemplated that each measurement portion may extend laterally anywhere 4.0 cm to 10 cm from each flank of the main body (8.0 cm and 20 cm in total width, respectively). The measuring means on the measurement portions may therefore show numerical markings and tick marks at 0.5 cm intervals from center of the main body, thereby providing a quantifiable reference of distance.

However, it is to be noted that any of the aforementioned dimensions are merely design considerations, and are not intended to be limiting. As can be appreciated by those in the art, many variations to the aforementioned dimensions may be made. For example: the measurement device may be manufactured from a variety of different flexible materials; the measurement device may be manufactured to be a wide range of arc lengths; the diameter of the holes in the main body may be larger or smaller, depending on the width; width and thickness of the main body may be varied to afford different degrees of flexibility and structural integrity; the reduced-width portions may be any width which is less than the rest of the main body; the measurement portions may range in width to aid in the location of electrode placement sites that are positioned off the “Z” Line and “Y” Line; and the measurement means may utilize any measure of distance at any reasonable increment.

Referring now to FIG. 1, in use, the holes in the main body of the measurement device may be used to identify the following electrode placement sites when fitted to a subject's head and placed along the “Z” Line: Fpz, Fz, Cz, Pz, and Oz. The holes in the main body of the measurement device may also identify the following electrode placement sites when placed along the “Y” Line: T4, C4, Cz, C3, and T3. Depending on their widths, the measurement portions may aid a worker in locating the following electrode placement sites which are positioned off the “Z” Line and “Y” Line: Fp1, Fp2, O1, O2, F7, F8, T5, and T6. After the user properly marks all of the appropriate electrode placement sites using the measurement device, he or she may obtain the remaining off-“Z” Line and off-“Y” Line electrode placement sites from the newly marked sites by using a tape measure and connecting two known points. The electrode placement sites that need to be located in this manner will ultimately depend on the total widths of the measurement portions.

While embodiments of this disclosure have been depicted and described and are defined by reference to exemplary embodiments of the disclosure, such references do not imply a limitation on the disclosure, and no such limitation is to be inferred. The subject matter disclosed is capable of considerable modification, alteration, and equivalents in form and function, as will occur to those skilled in the pertinent art and having the benefit of this disclosure. The depicted and described embodiments of this disclosure are examples only, and not exhaustive of the scope of the disclosure. For example, some alternative embodiments have been suggested to exemplify the versatility of the present disclosure but others may also be contemplated. Although the device is intended for use correlating to the International 10-20 system, it is understood that possible variations may exist depending on the specific standardized system practiced in the field. The device may be modified without departing from the scope of the invention. For instance, the device may be modified for an intended use correlating to the 10-10 system, 10-5 system, or any other system practiced in the field.

It should further be noted that throughout the entire disclosure, the labels such as left, right, front, back, top, bottom, forward, reverse, clockwise, counter clockwise, up, down, or other similar terms such as upper, lower, aft, fore, vertical, horizontal, oblique, proximal, distal, parallel, perpendicular, transverse, longitudinal, etc. have been used for convenience purposes only and are not intended to imply any particular fixed direction or orientation. Instead, they are used to reflect relative locations and/or directions/orientations between various portions of an object.

In addition, reference to “first,” “second,” “third,” and etc. members throughout the disclosure (and in particular, claims) are not used to show a serial or numerical limitation but instead are used to distinguish or identify the various members of the group.

Preferred embodiments are described herein, including the best mode known to the inventor. Of course, variations of those preferred embodiments will become apparent to those of ordinary skill in the art upon reading the foregoing description. The inventor expects skilled artisans to employ such variations as appropriate. Moreover, any combination of the above-described elements in all possible variations thereof is encompassed unless otherwise indicated herein or otherwise clearly contradicted by context.

What is claimed is:

1. A measuring device configured to aid in electrode placement for Electroencephalography tests or experiments comprising:
an elongated member having a fixed arc length, said elongated member including at least one measuring edge; and
at least five holes for electrode placement, extending along the length of said elongated member at fixed intervals.

2. The measuring device as claimed in claim 1, wherein said fixed arc length is greater than or equal to 15 centimeters.

3. The measuring device as claimed in claim 1, wherein said fixed arc length is less than or equal to 60 centimeters.

4. The measuring device as claimed in claim 1, wherein said five holes at said fixed intervals are located at 10%, 30%, 50%, 70%, and 90% of the said fixed arc length.

5. The measuring device as claimed in claim 1, wherein said measuring device is constructed from a non-rigid material.

6. A device for measuring locations on a scalp to place electrodes, the device comprising:
an elongated member having a fixed arc length; and
at least two holes for electrode placement.

7. The device as claimed in claim 6, wherein said fixed arc length is less than or equal to 60 centimeters.

8. The device as claimed in claim 6, wherein said at least two holes are located at 10% and 90% of said fixed arc length.

9. The device as claimed in claim 8, further comprising an additional set of holes at set intervals, said additional set of holes are located between the said at least two holes.

10. The device as claimed in claim 6, wherein said measuring device is constructed of a non-rigid material.

11. The measuring device as claimed in claim 10, wherein said measuring device is constructed from a thermoplastic material.

12. The measuring device as claimed in claim 6, wherein said elongated member comprises a set of measuring indicia with corresponding labels referencing American or metric units for said fixed arc length.

13. A measurement device for electrode scalp placement comprising:
a flexible body member comprising an elongated portion having a first width, a pair of distal ends having a second width, and at least one measurement portion having a third width; wherein said flexible body member includes a fixed arc length, and said at least one measurement portion includes a set of measuring indicia with corresponding labels referencing American or metric units for said fixed arc length;
a pair of holes for electrode placement, said a pair of holes positioned on said pair of distal ends.

14. The measurement device of claim 13, further comprising an additional set of holes at set intervals, said additional set of holes are located between said pair of holes on said elongated portion.

15. The measurement device as claimed in claim 13, wherein said fixed arc length is less than or equal to 60 centimeters.

16. The measurement device as claimed in claim 14, wherein each hole of said pair of holes is located at 10% and 90% respectively of said fixed arc length.

17. The measurement device as claimed in claim 14, wherein said additional set of holes are positioned at fixed intervals located at 30%, 50%, and 70%, of the said fixed arc length.

18. The measurement device as claimed in claim 13, wherein said flexible body member is constructed from a thermoplastic material.

19. The measurement device as claimed in claim 13, wherein said first width is greater than said second width and less than said third width.

20. The measurement device as claimed in claim 17, wherein said pair of holes and said additional set of holes correspond with an International 10-20 system of electrode placement.
